(19) United States
(12) Patent Application Publication
(10) Pub. No.: US 2007/0179587 A1
(43) Pub. Date: Aug. 2, 2007

(54) Apparatus and methods for deployment of custom-length prostheses

(75) Inventors: Pablo Acosta, Newark, CA (US); David Snow, San Carlos, CA (US)

Correspondence Address: TOWNSEND AND TOWNSEND AND CREW, LLP;(CLIENT NO 021629-000000), TWO EMBARCADERO CENTER, 8TH FLOOR, SAN FRANCISCO, CA, 94111-3834, US

(73) Assignee: Xtent, Inc., Menlo Park, CA (US)

(21) Appl. No.: 11/344,464

(22) Filed: Jan. 30, 2006

(51) Int. Cl. A61F 2/06

(52) U.S. Cl. 623/1.11

(57) ABSTRACT

A catheter for delivery of prosthetic stent segments comprises a separator tube having a one-way grip structure near a distal end thereof. The stent segments are delivered to a treatment region on a balloon. To select a number of distal stent segments for deployment, the separator tube is advanced distally over proximal stent segments. Proximal retraction of the separator tube pulls the engaged segment(s) proximally to separate proximal segments from distal segments, freeing the distal segments for deployment.

### BACKGROUND OF THE INVENTION

1. Field of the Invention

This invention relates generally to medical apparatus and methods, and more specifically to vascular catheters, stents and stent delivery catheters for deployment in the coronary arteries and other vessels.

Stenting has become an increasingly important treatment option for patients with coronary artery disease. Stenting involves the placement of a tubular prosthesis within a diseased coronary artery to maintain the patency of the artery, typically after a primary treatment such as angioplasty. Early stent technology suffered from restenosis, i.e., the tendency of the coronary artery to become re-occluded following stent placement. However, in recent years, restenosis rates have decreased substantially, due in part to drug coatings and other improvements in stent technology. As a result, the number of stent related procedures being performed in the United States, Europe, and elsewhere has increased dramatically.

Stents are delivered to the coronary arteries using long, flexible vascular catheters typically inserted through a femoral artery. For self-expanding stents, the stent is simply released from the delivery catheter and it resiliently expands into engagement with the vessel wall. For balloon expandable stents, a balloon on the delivery catheter is expanded which expands and deforms the stent to the desired diameter, whereupon the balloon is deflated and removed.

Current stent delivery technology suffers from a number of drawbacks which can make placement of prosthetic stents difficult. Of particular interest to the present application, current stent delivery catheters usually employ stents having fixed lengths. The proper selection of fixed length stents requires accurate knowledge of the length of the lesion being treated. While lesion length may be measured prior to stent deployment using angiography or fluoroscopy, such measurements are often inaccurate. Thus, if a stent is introduced that is found to be of inappropriate size, the delivery catheter and stent must be removed from the patient and replaced with a different device of correct size, which can take time and prolong the procedure.

The use of “custom length” stents as an alternative to fixed length stents has been proposed. One promising approach for providing a custom length stent has been to use segmented stents for treatment in which only some of the stents are deployed for treatment. As described in certain of the copending, commonly assigned applications listed below, the stent segments are deployed by selective advancement over the delivery catheter. After delivering an initial group of segments, the catheter may be repositioned and a further group of segments deployed. While a remarkable improvement over earlier technologies, to permit such segmental delivery, the delivery catheters can be somewhat complex and may require a larger diameter to accommodate the necessary structure.

Another difficulty with current stents which must be contended with is access to the stent delivery site. Blood vessels are not straight, and the surgeon or other person attempting to place a stent must often navigate blood vessels of the body with a catheter. Thus, a highly conformable (i.e. flexible) stent delivery catheter is desirable because such a catheter can bend and conform to the vessels of the human body. Diseased patients can have swollen or edematous tissues which can decrease the size of blood vessels used to access a lesion to be treated, thereby making access to a treatment region difficult. Also, prosthetic stent segments must be delivered through lesions which can occlude, at least partially and in some instances substantially, a vessel in which the prosthetic stent is delivered, illustrating the importance of profile and conformability. Thus, the size, profile and conformity of a deployment catheter can effect the success in accessing a lesion site.

For the above and other reasons, it would be desirable to provide improved prosthetic stents and stent delivery catheters. It would be particularly desirable to provide catheters and systems having simplified constructions and reduced crossing-profiles for delivering segmented stents where stent length can be reliably customized in situ as the stents are deployed.

2. Description of the Background Art

Prior publications describing catheters for delivering multiple segmented stents include: U.S. Publication Nos. 2004/0098081, 2005/0149159, 2004/0093061, 2005/0010276, 2005/0038505, 2004/0186551, 2004/0186551, and 2003/0135266. Prior related unpublished co-pending U.S. patent applications include Ser. No. 11/148,713, filed Jun. 8, 2005, (Attorney Docket No. 14592.4002), entitled “Devices and Methods for Operating and Controlling Interventional Apparatus”; Ser. No. 11/148,545, filed Jun. 8, 2005, (Attorney Docket No. 14592.4005), entitled “Apparatus and Methods for Deployment of Multiple Custom-Length Prosthesis”. The full disclosures of each of these patents and applications are incorporated herein by reference.

### BRIEF SUMMARY OF THE INVENTION

The invention generally provides for the delivery of stent segments with a low profile catheter which is flexible and conformable, especially the distal end. The low profile and conformable delivery catheter permits deployment of a selected number of the stent segments at a single site, thus permitting in situ customization of stent length to better match the length of the lesion being treated. The delivery catheter has a simplified design which grip structure for separating the selected group of stent segments prior to deployment.

In a first aspect, the invention comprises an apparatus for implanting prosthetic segments in a body lumen with a carrier, typically an elongated flexible carrier positionable in the body lumen. Such carriers are exemplified by conventional coronary, cerebral, and peripheral catheters of the type well described in the medical and patent literature. A plurality of prosthetic segments are axially distributed on an exterior surface of the carrier. The prosthetic segments are releasably secured or otherwise positioned on the exterior surface of the carrier so that they may be deployed in situ within the target body lumen. A separator is advanced distally over the segments and retracted proximally to separate a proximal group of the segments from a distal group of the segments. The separated distal group of segments can then be delivered into the body lumen while the remaining proximal stent segments remain constrained within the separator as described in detail below.

Usually, the selected distal group of prosthetic segments will be deployed by application of a radially outward internal force. The carrier comprises a catheter having an expandable member, typically an inflatable balloon. The expandable member provides the exterior surface which carries the plurality of prosthetic segments, and in an exemplary embodiment has a length in the range from 1 cm to 20 cm, and is expandable to a diameter in the range from 1 mm to 5 mm.

Optionally, the apparatus for implanting prosthetic segments of the present invention may further comprise a constraining tube or other structure which is positionable over. the inflatable balloon or other expandable member of the elongated flexible carrier. The constraining tube will have dimensions selected so that it can constrain or inhibit inflation of the balloon or expansion of other type of expandable member so that the length of expansion of the expandable member can be controlled. Typically, the constraining tube may form part of or otherwise be provided by the same structure as the separator which is used to separate proximal segments from distal segments, as described in more detail below.

The apparatus can include any number of prosthetic segments, for example from 2 to 50, usually from 2 to 30, and typically from 5 to 20 prosthetic segments carried by the expandable member. The prosthetic segments can have interleaved ends prior to axial separation, as described in co-pending, commonly assigned application Ser. No. 10/736,666, filed on Dec. 16, 2003, the disclosure of which is incorporated fully herein by reference. Such interleaved ends permit the segments to be packed closely on the carrier and provide a greater density of deployed prosthetic stent segments. The prosthetic segments typically each have a length in the range from 2 mm to 20 mm, more typically from 2 mm to 10 mm, and preferably from 4 mm to 8 mm.

In many embodiments, the separator comprises a separator tube having a distal end, a proximal end, a central passage, and an engagement member near the distal end thereof. The engagement member usually includes a grip structure which directly engages the distal most stent segment of the proximal group to be separated. The grip structure may be designed and fabricated so that it moves relatively freely over the plurality of stents as the separator tube is moved distally, but engages an adjacent stent segment when the separator tube is drawn proximally. Such grip structures which preferentially engage and apply a force to the adjacent stent segment are referred to hereinafter as “one-way grip structures.” By that, it is meant that they preferentially act to engage the adjacent stent segment only when pulled proximally. Other grip structures could be provided which engage and apply an essentially equal force to the adjacent or underlying stent segments as the separator tube is moved in both directions. In such cases, however, it will be necessary to prevent the plurality of stent segments from being moved distally as the separator tube is advanced thereover in a distal direction. For example, a nose cone or other distal structure may be provided on the elongated flexible carrier at a position immediately distal of the distal-most stent segment to prevent distal translation of the stent segments.

When using the exemplary one-way grip structure, the separator tube is advanced distally with the one-way grip structure passing over the stent segments, preferably exerting little or no force on the stent segments. After the grip is aligned with the distal most segment of the proximal group to be separated, the separator tube is retracted proximally, so that the one-way grip structure grips the distal most segment and draws the entire proximal group of segments proximally relative to the balloon or other exterior surface, thus separating the distal and proximal segment groups. The grip structure is typically spaced proximally from a distal end of the separator tube by a distance of about one-half to twice the length of a prosthetic segment, preferably being approximately equal to the length of a prosthetic segment. This setback of the grip structure provides a distal region of the separator tube, sometimes referred to hereinbelow as the “garage,” which will cover and constrain any portion of the distal-most stent segment which extends beyond the grip structure after separation of the proximal group of stent segments from the distal group of stent segments. Thus, regardless of where the grip structure engages, the distal-most stent segment along its length, little or none of that distal-most stent segment will extend distally outside of the separator tube so that the retracted stent segments are entirely contained within the separator tube during expansion of the selected distal segments. In such embodiments, the separator tube may comprise or otherwise provide all or a portion of the constraining tube referred to hereinbefore. The separator tube will be adapted to regularly restrain the retracted stent segments from expansion while the exposed distal segments are expanded.

A variety of one-way grip structures are useful in the apparatus of the present invention. For example, the one-way grip structure can include a multiplicity of radially inwardly extending resilient fingers, such as inclined resilient tabs formed in a metal ring. At least some of the fingers will usually be inclined proximally so that they will pass easily over the prosthetic segments as the separator tube is advanced distally but grip the adjacent segment when the separator tube is pulled proximally, thus acting as a “ratchet” mechanism. Alternatively, the one-way grip may include a balloon or other inflatable structure to permit selective engagement of the adjacent stent segment by inflation. In other embodiments the one-way grip is releasable so that the grip may be selectively engaged and released from the segments as the separator tube is advanced and/or retracted. The one-way grip could also include an inclined or conical surface. For example, a conical surface which tapers proximally to pass over the segments while advancing in a distal direction, and then grip the segments when the separator tube is retracted proximally. For example a conical surface can be arranged so that a smaller diameter trailing edge can be advanced distally over the stent segments. When retracted proximally, the edge will engage the adjacent segment to draw all proximal segments back proximally.

In another aspect, the invention comprises a method for delivering stent segments to a body lumen. A plurality of adjacent stent segments are introduced into the body lumen at or near a region to be treated. One or more distally positioned stent segments are selected for delivery to the body lumen. All of the stent segments which are located proximally of the selected stent segment(s) are axially separated from the distal stent segment(s). Any stent segments which are proximal to the selected stent segments are retracted proximally, usually simultaneously. The selected distal stent segment(s) are deployed after they have been separated from the proximally located stent segments.

In many embodiments, the plurality of adjacent stent segments are introduced into a blood vessel, for example to treat a lesion therein, typically following angioplasty or other primary interventional treatment. Angioplasty (predilation) or post dilation of the lesion can be performed by the catheter balloon of the present invention in the same intervention. The plurality of adjacent stent segments usually includes at least 3 stent segments, typically at least 5 stent segments, and often at least 10 stent segments. To facilitate separation of the stent segments, at least some of the adjacent stent segments are usually unattached prior to separating, for example unattached from each other and/or unattached from a surface of an expandable member. In other instances, at least some of the plurality of stent segments can be frangibly (or in other cases permanently) attached prior to separation or could be interconnected by biodegradable links which could erode and detach after implantation.

In many embodiments, deployment of the stent is performed while imaging the lesion, the catheter, and/or the stents real time. For example, the selection of the desired number of the stent segments can be performed under fluoroscopic imaging. The selection of the desired number of the stent segments can include aligning a marker disposed at or near the distal most stent segment with a distal end of a region to be treated and subsequently aligning a marker at or near the distal end of the separator tube with a proximal end of the lesion. The one-way grip or other engagement member is then properly aligned to separate a distal plurality of the stent segments having a length equal to that of the lesion. Inaccuracies resulting from imaging distortions, parallax errors, measurement errors, and/or catheter malpositioning are thus avoided.

In some embodiments, axial separation of the stent segments includes engaging the stent segment which is located immediately proximal of the selected segment(s) with a separator, and drawing the separator proximally. The separator can be a tube with a grip structure positioned near the distal end of the tube, and the grip structure is positioned over the immediately proximal stent segment to engage the immediately proximal stent segment. A deployment balloon or other expansible surface can be expanded to radially expand and deploy the selected stent segment(s). Generally, the proximally located stent segments are radially constrained, for example within the separator tube, while the selected stent segment(s) are radially expanded.

In yet another aspect, the invention comprises a method for selectively delivering stent segments to a treatment region in a blood vessel. A balloon deployment catheter is positioned through the blood vessel to the treatment region, and a plurality of adjacent stent segments are positioned over the balloon. A separator tube is advanced over one or more proximally positioned stent segment(s), and a grip structure on the separator tube engages against a distal most of the proximally positioned stent segments. The separator tube is pulled proximally to separate the proximally positioned stent segments from the remaining distally positioned stent segment(s). The balloon is inflated to deploy the distally positioned stent segment(s) while the proximally positioned stent segments remain covered by the separator tube.

The plurality usually includes at least 3 adjacent typically at least 5, and often at least 10 stent segments, and at least some of the plurality of stent segments are unattached prior to separation, so as to facilitate separation. Alternatively or in combination, at least some of the plurality of stent segments may be attached prior to separation to provide attached segments following deployment. The distal most stent segment can be aligned with the distal end of the region to be treated, and the grip structure engaged against a stent segment which lies immediately proximally of the proximal end of the region to be treated. The alignment can be performed with the aid of real time imaging, for example fluoroscopic imaging.

The particular aspects of the present invention described above may be employed in combination with a number of other features and capabilities of vascular and other stent structures and delivery systems. For example, the stents and other prosthetic segments of the present invention may be covered with drugs and bioactive agents, such as anti-restenotic agents as well described in the co-pending applications previously incorporated herein by reference. In other instances, the prosthetic and stent segments could be formed from a shape or heat memory alloy and be self-expanding. In such cases, the stent segments could be carried on the inside surface of the constraining tube where the separator would be coaxially received within the restraining tube. The stent and prosthetic segments could also be formed from bioresorbable materials, and would be useful in a wide variety of vascular and non-vascular body lumens. Vascular body lumens include the coronary, peripheral, and cerebral vasculature. Non-vascular body lumens include the ureter, urethra, fallopian tubes, spinal column, and the like.

### BRIEF DESCRIPTION OF THE DRAWINGS

FIG. 1A shows a perspective view of a stent delivery catheter with an outer separator tube retracted and an expandable member inflated, in accordance with the present invention.

FIG. 1B shows a fully retracted separator, exposed prosthetic segments disposed over an expandable member, and a one-way grip structure in accordance with and embodiment.

FIGS. 2A-2D show deployment of selected prosthetic segments to treat a lesion in accordance with an embodiment.

FIG. 3A shows a one-way grip structure which includes a brake release.

FIG. 3B shows a stent retention tube used to retain prosthetic segments.

FIG. 4A shows a one-way grip structure which includes a deflectable flange or prong.

FIG. 4B shows a one-way grip structure which includes an “L” shaped deflectable flange or prong.

FIG. 4C shows a one-way grip structure which includes an annular inflatable balloon.

FIG. 4D shows a one-way grip structure which includes a unilateral inflatable balloon.

FIG. 4E shows a one-way grip structure which includes a flange or O-Ring.

FIG. 5A shows a one-way grip structure which includes shape memory using a Ni/Ti cylinder or wire loop

FIG. 5B shows a one-way grip structure which includes flexible saw teeth or threads.

FIG. 5C shows a one-way grip structure which includes bristles, foam or fabric.

FIG. 5D shows a one-way grip structure which includes a tapered flange.

FIG. 6 shows a garage located at the end of the stent separator tube in which the garage includes one-way grip structures in accordance with an embodiment.

FIG. 7 shows another garage located at the end of the stent separator tube in accordance with an embodiment.

FIG. 8 shows a garage as in FIG. 7 having concave arcuate cutouts on the ends of rectangular flanges which engage the prosthetic segments.

FIGS. 9A and 9B show plan and perspective views of a one-way grip structure with an arcuate flange in accordance with an embodiment.

### DETAILED DESCRIPTION OF THE INVENTION

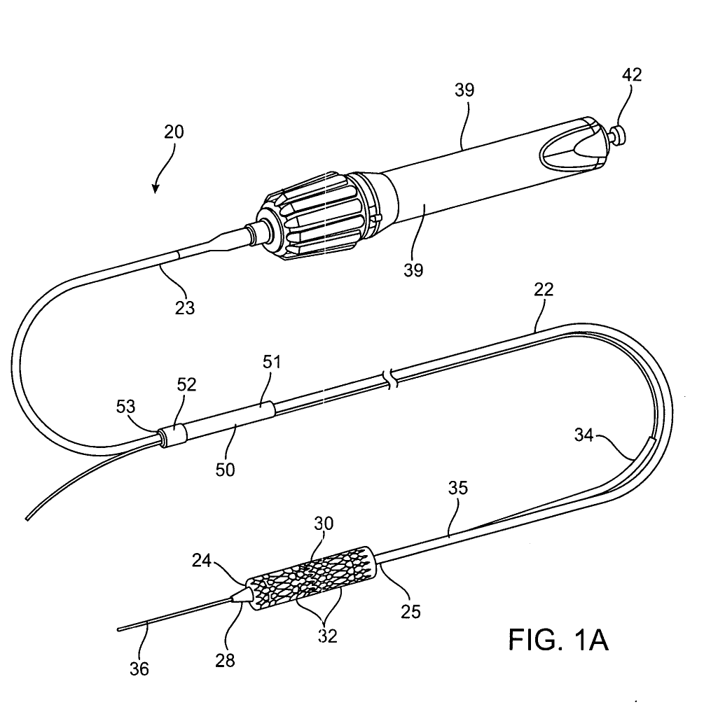

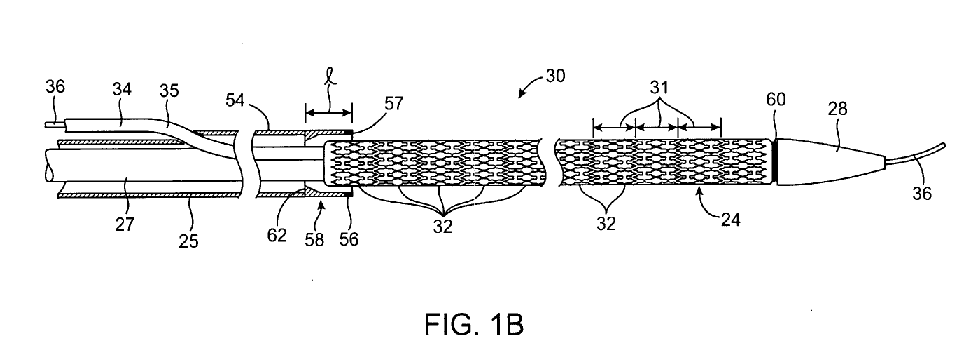

Referring now to FIGS. 1A and 1B, a stent delivery catheter 20 includes an elongated flexible carrier, such as a catheter body 22, an inner inflation shaft 27, or a separator tube 25 slidably disposed over the inner inflation shaft 27 (FIG. 1B). An expandable member 24, usually an inflatable balloon (shown in an inflated configuration in FIG. 1A and a deflated condition in FIG. 1B), is mounted at a distal end of inner inflation shaft 27 and is exposed by retracting the separator tube 25 relative to inner shaft. Inner shaft 27 includes a lumen which is fluidly connected to inflatable member 24. A guidewire tube 34 is slidably positioned through a guidewire tube exit port 35 in separator tube 25 proximal to expandable member 24. Guidewire tube 34 extends through the interior of expandable member 24, the distal end of which is sealingly attached to guidewire tube 34. The proximal end of expandable member 24 is sealingly affixed around guidewire tube 34 and inner shaft 27. A tapered nosecone 28, typically composed of a soft elastomeric material, is mounted to guidewire tube 34 distally of expandable member 24 to reduce trauma to the vessel during advancement of the device. A prosthesis 30, which comprises a plurality of separate or separable prosthetic segments 32, is disposed on expandable member 24 for expansion therewith. A guidewire 36 is positioned slidably through guidewire tube 34, expandable member 24, and nosecone 28 and extends distally thereof.

A handle 38 is attached to a proximal end 23 of catheter body 22. Handle 38 performs several functions, including advancing and retracting the separator tube, connecting a balloon inflation source, manipulating the catheter, etc. Various embodiments of a preferred handle and additional details concerning its structure and operation are described in co-pending U.S. patent application Ser. No. 11/148,713, filed Jun. 8, 2005, (Attorney Docket No. 14592.4002), entitled “Devices and Methods for Operating and Controlling Interventional Apparatus,” which application has been previously incorporated herein by reference. Embodiments of another preferred handle and details concerning its structure and operation are described in co-pending U.S. Publication No. 2005/0149159, entitled “Devices and Methods for Controlling and Indicating the Length of an Interventional Element,” which application has previously been incorporated herein by reference.

Handle 38 includes a housing 39 that encloses the internal components of the handle. Inner shaft 27 is preferably fixed to the handle, while separator tube 25 is able to be retracted and advanced relative to the handle 38. An adaptor 42 is attached to handle 38 at its proximal end, and is fluidly coupled to inner shaft 27 in the interior of the housing of handle 38. Adaptor 42 is configured to be fluidly coupled to an inflation device, which may be any commercially available balloon inflation device such as those sold under the trade name “Indeflator™”, available from Guidant Corp. of Santa Clara, Calif. The adaptor is in fluid communication with expandable member 24 via an inflation lumen in inner shaft 27 to enable inflation of expandable member 24.

Separator tube 25 and guidewire 36 each extend through a slider assembly 50 located on the catheter body 22 at a point between proximal and distal ends of the catheter body. Slider assembly 50 is adapted for insertion into and sealing within a hemostatic valve (not shown), such as on an introducer tube or guiding catheter, while allowing relative movement of separator tube 25 relative to slider assembly 50. Slider assembly 50 includes a slider tube 51, a slider body 52, and a slider cap 53.

Referring now to FIG. 1B, the separator tube 25 is shown fully retracted to expose the plurality of prosthetic segments 32 which are disposed over expandable member 24. Expandable member 24 acts as a carrier which supports the prosthetic segments. Separator tube 25 includes an engagement member 58, such as a one-way grip structure 62. In some embodiments, described more fully herein below, a distal region 54 of the separator tube defines a garage for covering and constraining a portion of a prosthetic segment which extends beyond the grip 62 after separation. Separator tube 25 and engagement member 58 may be advanced toward nosecone 28 in a distal direction relative to expandable member 24 and stent segments 32. Each of the stent segments 32 has an axial length, typically from 1 mm to 50 mm, usually about 2 mm to 20 mm, and preferably about 3 mm to 10 mm.

Grip structure 62 is typically located within a distance λ relative to distal end 57, where λ is typically from one-half to twice the stent segment length, more preferably from about one to 1.5 times the segment length. In exemplary embodiments λ will be about 3 mm to 10 mm, with longer lengths being associated with longer segment lengths. Grip structure 62 contacts and engages stent segments 32. A distal portion 54 of separator tube 25 has a high circumferential strength, or hoop strength, such that the distal portion of the separator tube is able to prevent the expandable member 24 from expanding when the separator tube is extended over expandable member 24. The distal portion 54 of the separator tube 25 is preferably formed from metal or a polymer reinforced with a metallic or polymeric braid to resist radial expansion when expandable member 24 is expanded. Separator tube 25 may further have a liner surrounding its interior of lubricious or low friction material such as PTFE to facilitate relative motion of separator tube 25.

The one-way grip structure 62 provides certain advantages. For example, a “one-way” grip structure can be designed to apply greater force when separator tube 25 is retracted proximally in order to reliably separate the stent segments without slippage.

Radiopaque markers are preferably provided on the catheter to assist in positioning the catheter relative to the lesion and in selecting stent segments for deployment. A first radiopaque marker 56 (referred to as the “tube marker”) is disposed at the distal end 57 of the separator tube 25 to facilitate visualization of the position of separator tube 25. A second radiopaque marker 60 is disposed on the inner shaft 27 near the distal end of expandable member 24. The second marker 60 may be referred to as the “balloon marker.”

The distance between a first marker 56 and second marker 60 will correspond to the length of prosthetic segments 32 which are exposed for deployment after the separator tube 25 has been drawn proximally to pull back the proximal groups of segments which are not being deployed. Thus, by aligning the markers 56 and 60 with the two ends of the lesion being treated, the physician can assure that the deployed prosthesis length closely matches the lesion length being treated. This is a particular advantage when the apparent lesion length is foreshortened due to the tortuosity and viewing angle in the fluoroscopic image.

Grip structure 62 will usually be spaced proximally from distal end 57 of separator tube 25 by a distance sufficient to leave a distal “overhang” which will cover any portion of the distal most prosthetic segment which extends beyond the grip. For example, grip structure 62 can be spaced proximally from distal end 57 a distance λ approximately equal to axial length 31 of one of stent segments 32. In a preferred embodiment, each of stent segments 32 has the axial length of about 4 mm, and the grip structure 62 is located approximately 4 mm from distal end 57. In other embodiments, grip structure 62 may be positioned at distal end 57 a distance or spaced proximally any distance up to twice or more times In an embodiment using ten stent segments positioned on the catheter, from one to ten stent segments 32 can be deployed, and each of the ten stent segments can have an equal axial length 31. In a preferred embodiment, each of stent segments 32 are identical. Each of stent segments 32 can have interleaved ends in which a proximal end of a distal stent segment meshes with a distal end of an adjacent and proximally located stent segment as shown in FIG. 1B. Such interleaving ensures adequate wall coverage and reduces or eliminates gaps between segments after deployment in the body lumen being treated.

As shown in FIG. 1B, one-way grip structure 62 includes a necked-down circumferential waist or inwardly extending annular flange structure configured to frictionally engage stent segments 32 and thereby restrict the sliding movement of separator tube 25 relative to stent segments 32 when separator tube 25 is being retracted. One-way grip structure 62 may be a polymeric or metallic material integrally formed with separator tube 25 or may be bonded or otherwise mounted to the interior of the separator tube 25. The geometry of one-way grip structure 62 may be toroidal with a circular or ovoid cross-section (like an O-ring) or the grip structure may have another cross-sectional shape such as triangular, trapezoidal, pyramidal, or other shape as described more fully herein below. One-way grip structure 62 can be a polymer such as silicone or urethane sufficiently soft, compliant, and resilient to provide frictional engagement with stent segments 32, in some embodiments without damaging any coating deposited thereon. Grip structure 62 will extend radially inwardly a sufficient distance to engage the exterior of stent segments 32 with sufficient force to allow the line of stent segments 32 remaining within separator tube 25 to be retracted proximally with separator tube 25 so as to create spacing relative to those stent segments disposed distally of separator tube 25 for deployment.

Any desired number of segments 32 can be used, and segments 32 may have a wide variety of lengths. In a preferred embodiment, balloon 24 has a length in the range from 60 mm to 65 mm, and up to fifteen 4 mm stent segments 32 can be deployed over a maximum deployment distance of up to 60 mm. In alternative embodiments, up to ten 6 mm stent segments can be deployed over a maximum deployment distance of up to 60 mm. The stent segments can be crimped onto the expandable member 24 so that the expandable member carries the stent segments. In some embodiments, the maximum deployment distance can be up to 200 mm or greater, and in further embodiments the inflatable member can be a tapered balloon to enhance stability of stent segments 32, particularly where lesion 70 is long. For example, expandable member 24 can be tapered from an inflated outer diameter of 2.5 mm at its distal end to an outer diameter of about 3 mm at its proximal end.

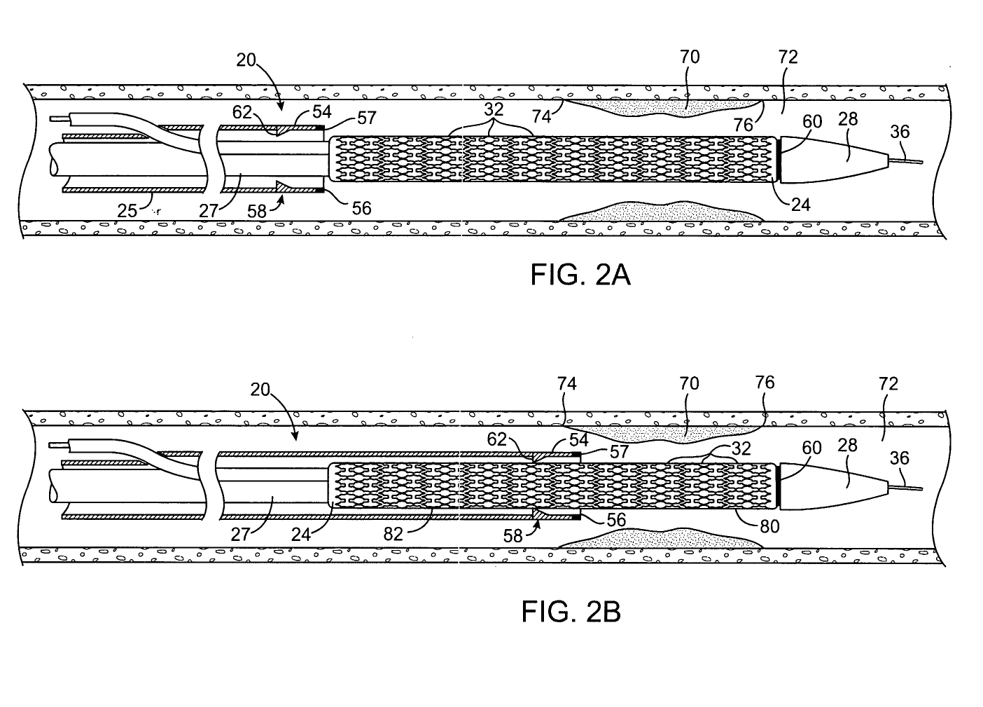

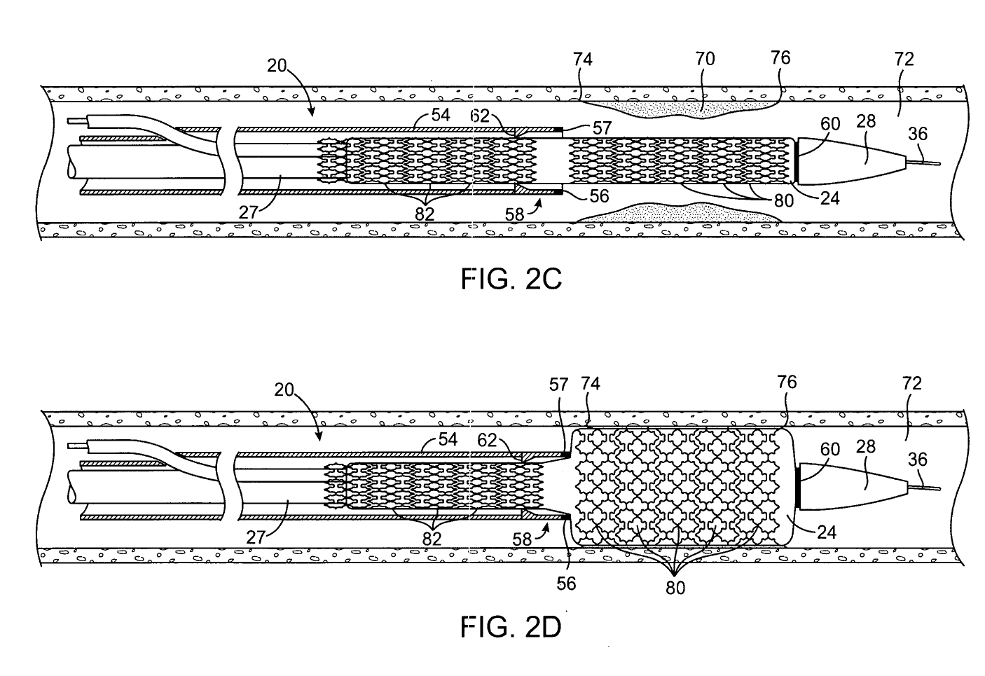

Referring now to FIGS. 2A-2D, the deployment of selected prosthetic segments to treat a lesion is shown in accordance with an exemplary embodiment. While the embodiment will be described in the context of coronary artery treatment, it should be understood that the invention is useful in any of a variety of blood vessels and other body lumens in which stents are deployed, including the carotid, femoral, iliac and other arteries and veins, as well as non-vascular body lumens, such as the ureter, the urethra, fallopian tubes, the hepatic duct, and the like. A guiding catheter (not shown) is first inserted into a peripheral artery such as the femoral and advanced to the ostium of the right or left coronary artery. Guidewire 36 is then inserted through the guiding catheter and advanced into the target coronary artery 72 where a lesion 70 is to be treated. A region to be treated, for example lesion 70, is bounded by a proximal end 74 and a distal end 76. The proximal end of guidewire 36 is then inserted through nosecone 28 and guidewire tube 34 outside the patient's body and stent delivery catheter 20 is slidably advanced over guidewire 36 into the coronary artery. Slider assembly 50 is positioned within the hemostasis valve at the proximal end of the guiding catheter, which is then tightened to provide a hemostatic seal with the exterior of the slider body 52. Stent delivery catheter 20 is positioned through lesion 70 to be treated such that nosecone 28 is distal to lesion 70. Marker 60 is positioned near distal end 76 of lesion 70. During this positioning, separator tube 25 is retracted proximally so as to expose expandable member 24 and all of the stent segments 32 thereon as shown in FIG. 2A. Use of the retracted separator tube during positioning of delivery catheter 20 can have the advantage of presenting a lower profile catheter to improve delivery to the lesion site, and presenting a highly flexible and conformable catheter in the distal portion of the catheter, which are particularly advantageous when passing through tortuous lumens.

Referring now to FIG. 2B, separator tube 25 is advanced distally over the segments until marker 56 is positioned near proximal end 74 of the treatment region so as to permit removal of a proximal group 82 of segments 32 which are not needed to treat lesion 70. A desired treatment distance corresponding to a desired number of deployed stent segments can be determined by a separation distance between the first marker 56 on separator tube 25 and second radiopaque marker 60 adjacent nose cone 28. As separator tube 25 advances, one-way grip structure 62 advances over proximal stent segments 82. Distal stent segments 80 are located distal to one-way grip structure 62, and are selected for deployment based on the separation distance between the radiopaque markers. In some circumstances, such as when the catheter is positioned in a tightly curved or tortuous region of a vessel, stent segments 32 may tend to flare outwardly at their proximal ends, which may hamper advancement of separator tube 25. To address this, in some embodiments the distal end of separator tube 25 (or garage 54) may flare outwardly or may have an inner diameter that tapers in the proximal direction so as to present a larger diameter distal opening to receive stent segments 32 as the separator tube is advanced.

Referring now to FIG. 2C, once separator tube 25 has advanced over the proximal segments, the separator tube is retracted slightly to create a space between distal stent segments 80 and proximal stent segments 82. This space reduces the risk of dislodging or partially expanding the distal-most one of stent segments 82 when expandable member 24 is expanded. Usually, it is preferred to create a space of about 1 to 5 mm between the stent segments to be deployed and those remaining enclosed within the separator tube 25. Retraction of separator tube 25 causes one-way grip structure 62 to grip and retract proximal stent segments 82 so as to separate proximal stent segments 82 from deployed stent segments 80. Deployed stent segments 80 are uncovered and remain adjacent to lesion 70.

As shown in FIG. 2D, expandable member 24 is filled with fluid to expand radially so as to deploy distal stent segments 80. Radial expansion of deployed stent segments 80 urges deployed stent segments 80 outward against the vessel wall across lesion 70. Separator tube 25 constrains inflatable member 24 and prevents deployment of proximal stent segments 82. The number of stent segments 32 which are deployed will usually correspond to total stent or prosthesis lengths in the range from 4 to 200 mm. After stent segments 80 are deployed, inflatable member 24 is deflated and removed from deployed stent segments 80, leaving deployed stent segments 80 in a plastically-deformed, expanded configuration. Catheter 20 can then be removed and retracted from coronary artery 72.

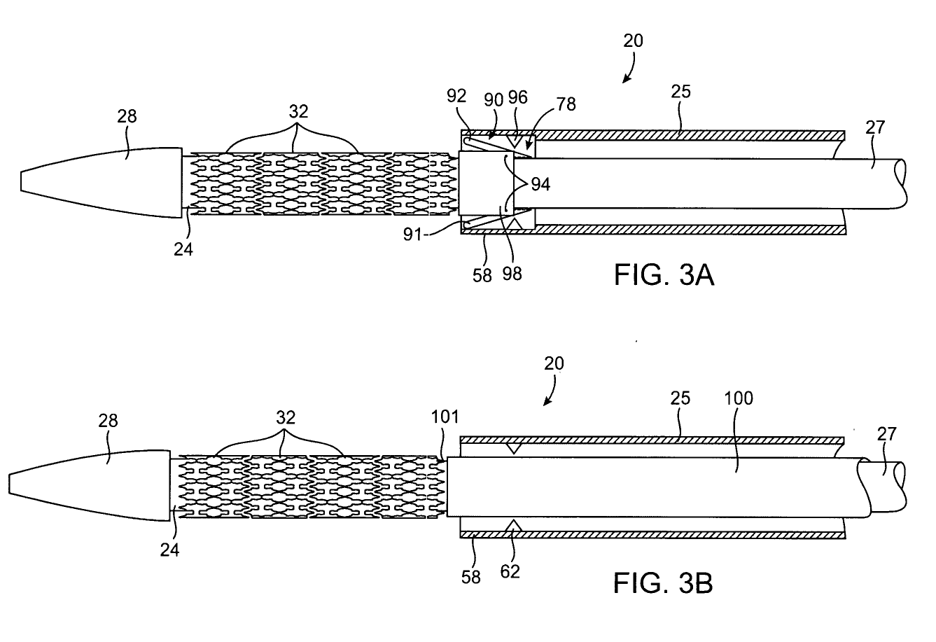

Referring now to FIG. 3A, engagement member 58 includes a one-way grip structure 78 with a brake release 90 which holds stent segments 32 in place on expandable member 24 during introduction of the catheter 20 into a body lumen. The brake release 90 includes a pair of arms 92 which pivot about attachment pins 94. Each attachment pin 94 is coupled to a tubular slide 98 which slides over the inner inflation shaft 27. Arms 92 and pins 94 are mounted to move with slide 98, and grip structure 96 is disposed on separator tube 25. Proximal to the grip structure 96, the separator tube 25 has a proximal portion 97 with a reduced inner diameter. Advancement of the separator tube 25 causes the grip or wedge 96 to engage arm 92 which in turn pivots the arm about pin 94 to disengage the arm from the underlying shaft. The outer separator tube can be advanced by a desired distance to select a desired number of prosthetic or stent segments for deployment. The reduced inner diameter of the proximal portion of the separator tube 25 keeps arms 92 disengaged. Once the outer separator tube 25 has been advanced a desired distance, separator tube is retracted proximally. Because arms 92 are disengaged from inner shaft 27, the slide 98 is able to move proximally as the separator tube 25 is retracted. Thus, the proximal segments are allowed to separate from the distal segments being deployed. The separated distal segments may then be deployed as described above.

Referring now to FIG. 3B, a stent retention tube 100 can be used to retain prosthetic segments 32 on an expandable member 24 during delivery to a treatment region. Stent retention tube 100 is disposed slidably over shaft 27 and within separator tube 25. Stent retention tube 100 has a distal end 101 that engages stent segments 32 and holds the segments in place relative to expandable member 24. Separator tube 25 can be advanced distally relative to the stent retention tube 100 in order to cover a desired number of stent segments which will not be deployed. The separator tube 25 and the stent retention tube 100 are together retracted proximally to separate deployed segments from proximal segments as described above. It should be understood that when the movement of the stent retention tube, separator tube, or stent segments is described in relation to other components of the delivery catheter, such movement is relative and will encompass moving the separator tube, stent retention tube, or stent segments while keeping the other component(s) stationary; keeping the separator tube, stent retention tube or stent segments stationary while moving the other component(s); or moving multiple components simultaneously relative to each other.

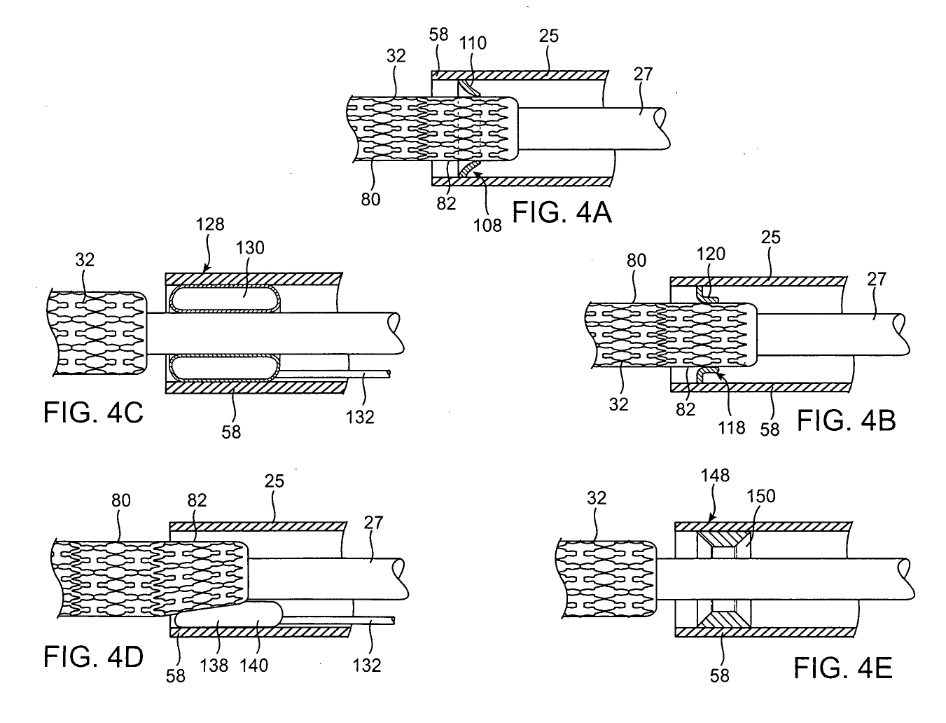

Referring now to FIG. 4A, engagement member 58 can include a one-way grip structure 108 with a deflectable flange 110 or prong. Deflectable flange 110 extends inward to engage unused proximal stent segment(s) 82. Deflectable flange 110 is resilient and may be inclined proximally to pass over proximal stent segments 82 as the separator tube advances distally. Separator tube 25 is advanced distally as described above to select stent segments for deployment. Proximal retraction of separator tube 25 engages the most distal of the proximal stent segments 82 with deflectable flange 110, and the proximal stent segments are retracted as described above.

Referring now to FIG. 4B, engagement member 58 can include a one-way grip structure 118 with a plurality of deflectable prongs 120 arranged around the inner circumference of separator tube 25. The resilient and deflectable prong bends proximally as separator tube 25 is advanced relative to stent segments 32.

Referring now to FIG. 4C, engagement member 58 can include a one-way grip structure 128 with an annular inflatable balloon 130. Annular balloon 130 is deflated and inflated using a lumen 132. To select stents for deployment, annular balloon 130 is first deflated, or initially provided in a deflated state. Deflated annular balloon 130 is positioned over the distal most of the proximal stent segments to select stents for deployment as described above. Annular balloon 130 is inflated to engage the distal most of the proximal stent segments. Proximal retraction of separator tube 25 as described above retracts the proximal stent segments to separate the distal stent segments for deployment. The distal stent segments are then deployed as described above.

Referring now to FIG. 4D, engagement member 58 includes a one-way grip structure 138 with a unilateral inflatable balloon 140. Unilateral balloon 140 can be used in a manner similar to annular balloon 130 as described above.

Referring now to FIG. 4E, engagement member 58 can include a one-way grip structure 148 with a flange 150 or O-Ring. Flange 150 can be made from a polymeric material, or metal such as a Nickel/Titanium alloy as described above. Use of flange 150 is similar to other one-way engagement members described herein. For example, flange 150 can be moved distally to slide over and cover exposed segments as described above. Flange 150 frictionally engages stent segments 32 such that upon retraction, flange 150 separates the proximal segments from the deployed segments as described above.

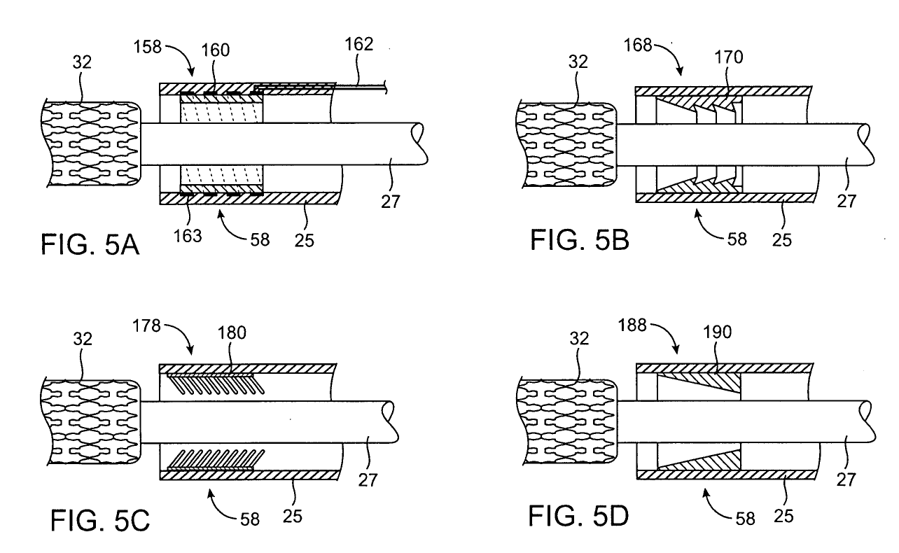

Referring now to FIG. 5A, engagement member 58 can include a one-way grip structure 158 with a shape memory member structure comprising a Ni/Ti cylinder 160 surrounded by a heating coil 163. Voltage and/or current is applied to heating coil 163 with wires. Prior to application of voltage and/or current, cylinder 160 has a large diameter that may be positioned over segments 32. Application of voltage and/or current to heating coils 163 causes a Ni/Ti cylender 160 to contract in diameter and engage segments 32. Separator tube 25 is retracted proximally to remove proximal segments and leave distal segments in position for deployment as described above.

Referring now to FIG. 5B, engagement member 58 can include a one-way grip structure 168 with flexible saw teeth 170 or threads. Flexible teeth 170 or threads can be rigid or flexible metal or polymer used in a manner similar to that described above with respect to the use of the flange.

Referring now to FIG. 5C, engagement member 58 can include a one-way grip structure 178 with bristles 180, or a foam or fabric material. Bristles 180, foam or fabric can be deployed in a manner similar to the flange described above.

Referring now to FIG. 5D, engagement member 58 can include a one-way grip structure 188 with a tapered flange 190. Tapered flange 190 is tapered to have a smaller diameter at its proximal end and be suitable for one-way engagement of segments 32.

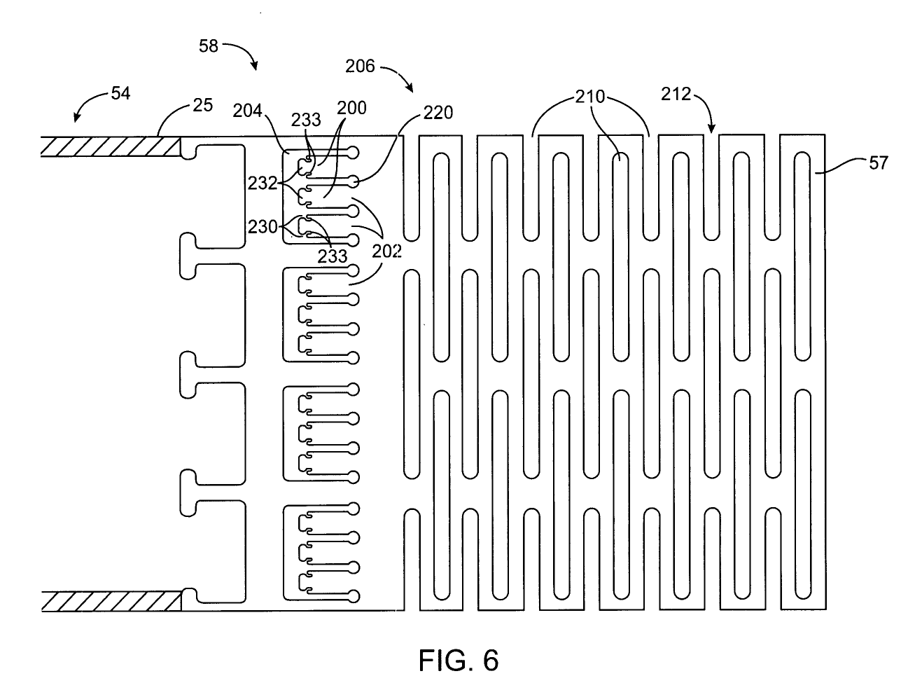

Referring now to FIG. 6, engagement member 58 can include a garage 206 having a plurality of one-way grip structures 204 formed thereon. Each one-way grip structure 204 includes several resilient tabs 200, or fingers, which can be angled inward and proximally inclined to engage segments 32 as described above. Tabs 200 can include a repeating pattern of three adjacent fingers. Each tab can include a rounded end 232 to avoid damage to a coating on the engaged stent segment. Circular cutouts 220 can distribute forces from tabs 200 which are applied to adjacent region 202 to prevent tearing of the garage. Also, a cross sectional size of circular cutouts 220 can be varied to provide resiliency and vary an amount of pressure which tabs 200 apply to the stent segments. Recesses 230 can be provided near rounded end 232 of the tabs 200. Recesses 230 can be provided on either side of rounded end 232 so as to define a pair of tips 233 along the lateral sides of the tabs 200. Tips 233 are adapted to engage the stent segments so as to keep rounded ends 232 from digging into expandable member 24 as the garage 206 is retracted relative to expandable member 24.

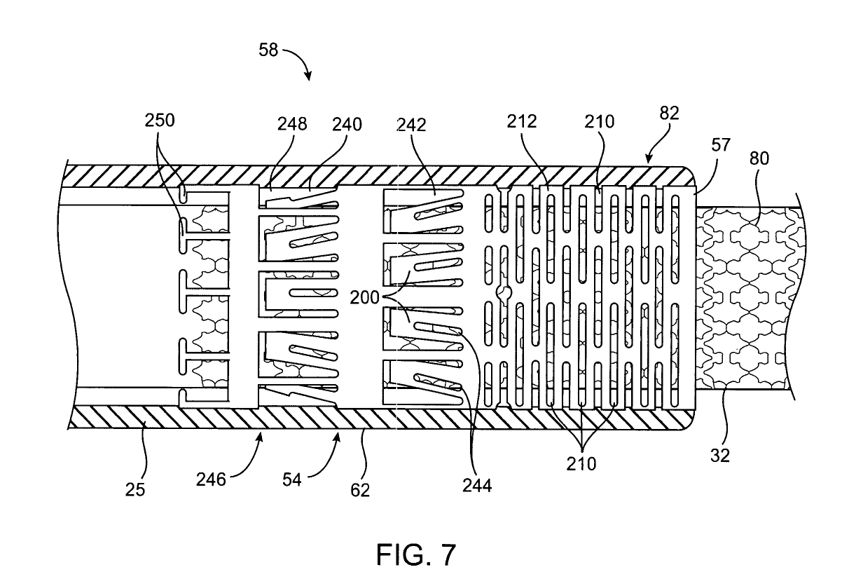

Garage 206 is generally cylindrical and is fixed to distal end 57 of separator tube 25. Garage 204 preferably has a length at least as long as one of stent segments 32, but preferably less than a combined length of two such stent segments. Garage 206 has channels 210 formed to provide a flexible body 212 and permit flexure of the garage during insertion of the catheter toward the treatment region. As shown in FIG. 6, garage 204 is attached to distal portion 54 of separator tube 25 so as to define distal end 57 of separator tube 25. As distal end 57 of separator tube 25, garage 204 is designed to have a high radial strength to prevent the expandable member 24 from expanding substantially when the garage is extended over inflatable member 24. Alternatively, the garage can be embedded within a distal portion 54 of separator tube 25 (see FIGS. 7, 8 and 9A below). Garage 204 can be made from any suitable material, or combination of suitable materials, for example Nickel/Titanium alloy or steel. Garage 204, tabs 200 and channels 210 can be formed by laser cutting or lithographic techniques such as photoetching. Garage 204 can be formed by mating ends of a photo etched flat plate to form a rounded cylinder. Alternatively, garage 204 can be formed from Ni/Ti alloy formed as a round tube which is laser machined.

Referring now to FIG. 7, engagement member 58 can include a garage 246 within a distal portion 54 of separator tube 25. Garage 246 includes a one-way grip structure 248 comprising two axially displaced rows of a repeating pattern of resilient tabs 200, or fingers. The tabs can be rectangular shaped to engage prosthetic segments 32. A first row of tabs 200 and a second row of tabs 200 are shown but one, three, or more rows could also find use. Elongate cutouts 244 are provided in the tabs 200 to decrease and/or set to desired gripping characteristics exerted on segments 32 by tabs 200. Anchors 250 are located on garage 246. Anchors 250 secure garage 246 to distal portion 54 of separator tube 25.

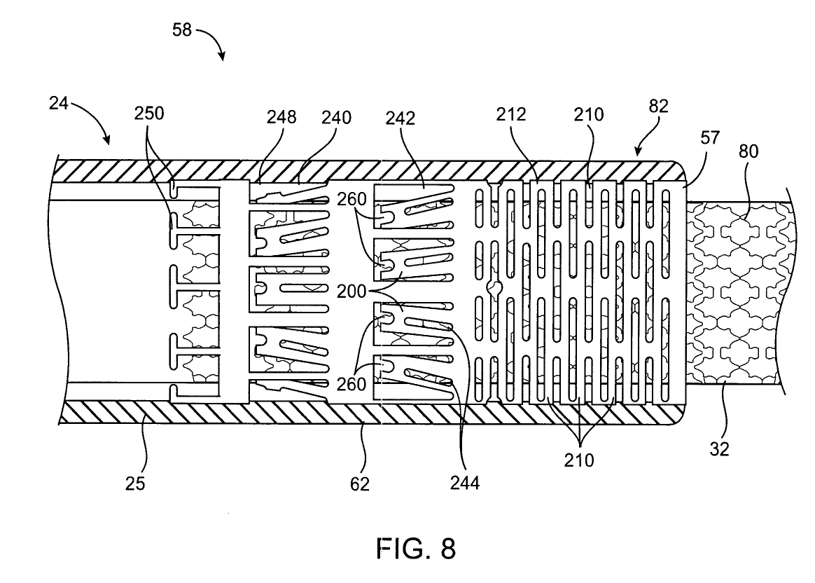

Referring now to FIG. 8, in a further embodiment, the garage and one-way grip structures as in FIG. 7 can have concave arcuate cutouts 260 on the ends of the rectangular tabs 200 which engage the prosthetic segments. Such cutouts 260 enhance engagement of the tabs 200 with the stent segments 32.

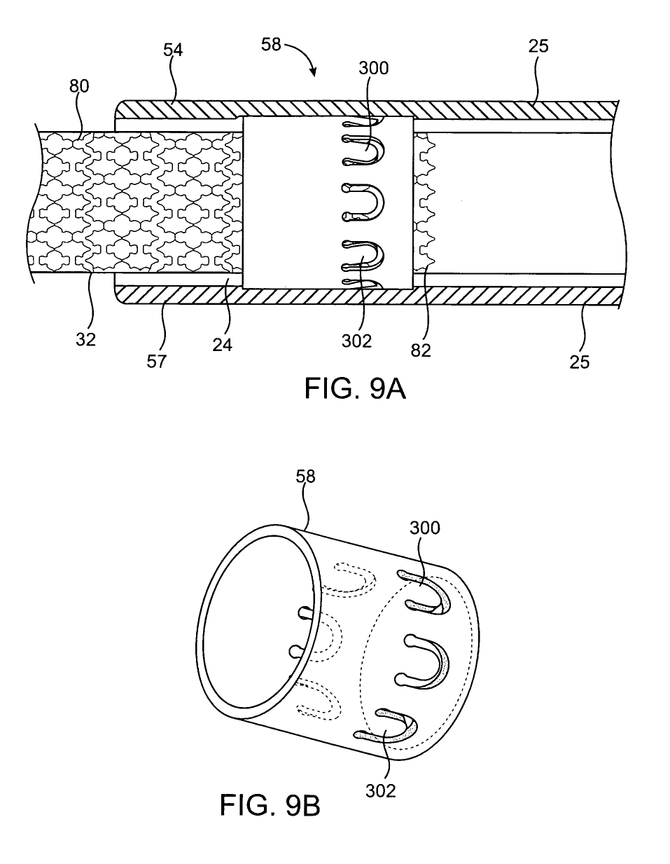

Referring now to FIGS. 9A and 9B, plan and perspective views are shown of engagement member 58 which includes a one-way grip structure 300 having an arcuate, resilient flange 302, or finger, in accordance with an embodiment. One-way grip structure 300 is located near distal end 57 of separator tube 25. One-way grip structure 300 is located within high strength distal portion 54 of the separator tube 25 so that the distal portion of the tube supports the grip structure and prevents expansion of the expandable member as described above. One-way grip structure 300 is separated from distal end 57 of the separator tube, and can be separated by any distance as described above. One-way grip structure 300 can be used, manufactured and machined similar to the garages described above.

While the exemplary embodiments have been described in some detail for clarity of understanding and by way of example, a variety of additional modifications, adaptations, and changes may be clear to those of skill in the art. Hence, the scope of the present invention is limited solely by the appended claims.

What is claimed is:

1. Apparatus for implanting prosthetic segments in a body lumen, said apparatus comprising:
an elongated flexible carrier positionable in a body lumen;
a plurality of prosthetic segments axially and releasably distributed on an exterior surface of the carrier; and
a separator which can be advanced distally over the segments and retracted proximally to separate a proximal group of the segments from a distal group of the segments which are to be released to the body lumen.

2. Apparatus as in claim 1, wherein the prosthetic segments expand upon application of a radially outward internal force, and the elongated flexible carrier comprises a catheter having an expandable member comprising the exterior surface which carries the plurality of prosthetic segments.

3. Apparatus as in claim 2, wherein the expandable member has a length in the range from 1 cm to 20 cm and is expandable to a diameter in the range from 1 mm to 5 mm.

4. Apparatus as in claim 3, having from 3 to 50 prosthetic segments carried by the expandable member.

5. Apparatus as in claim 2, wherein the expandable member comprises an inflatable member.

6. Apparatus as in claim 1, wherein the prosthetic segments have interleaved ends prior to axial separation.

7. Apparatus as in claim 1, wherein the prosthetic segments each have a length in the range from 2 mm to 20 mm.

8. Apparatus as in claim 1, wherein the separator comprises a separator tube having an engagement member near a distal end of the separator tube.

9. Apparatus as in claim 8, wherein the separator comprises a separator tube that surrounds a proximal group of segments after the separator has been distally advanced.

10. Apparatus as in claim 8, wherein the engagement member comprises a grip structure that passes over the prosthetic segments when the separator tube is advanced distally and which grips a prosthetic segment as the separator tube is pulled proximally.

11. Apparatus as in claim 10, wherein the grip structure exerts greater force against the prosthetic segment as the separator tube is pulled proximally than when the separator tube is pushed distally.

12. Apparatus as in claim 10, wherein the grip structure is spaced proximally from the distal end of the separator tube by a distance equal to about one-half to twice the length of a prosthetic segment.

13. Apparatus as in claim 10, wherein the grip structure comprises a multiplicity of radially inwardly located resilient fingers.

14. Apparatus as in claim 13, wherein at least some of the fingers are inclined proximally so that they will pass over the prosthetic segments as the separator tube is advanced distally but will engage a segment when the separator tube is pulled proximally.

15. Apparatus as in claim 14, wherein at least some of the fingers are composed of a metal.

16. Apparatus as in claim 10, wherein the grip structure is inflatable.

17. Apparatus as in claim 10, wherein the grip structure is releasable.

18. Apparatus as in claim 17, wherein the grip structure is adapted to selectively engage and disengage the prosthetic segments.

19. Apparatus as in claim 10, wherein the one-way grip structure comprises an inclined or conical surface.

20. Apparatus as in claim 19, wherein the grip structure comprises an annular flange on the separator tube.

21. Apparatus as in claim 1, wherein the prosthetic segments carry a therapeutic agent adapted to being released therefrom.

22. The apparatus of claim 21, wherein the therapeutic agent inhibits restenosis.

23. Apparatus as in claim 21, wherein the therapeutic agent is coated over at least a portion of the surface of the prosthetic segments.

24. A method for delivering stent segments to a body lumen, said method comprising:
introducing a plurality of adjacent stent segments at a region of the body lumen to be treated;
selecting one or more distally positioned stent segments for delivery to the body lumen;
axially separating all stent segments located proximally of the selected distally positioned stent segment(s) from the selected distally positioned stent segment(s); and
deploying the selected distally positioned stent segment(s) after they have been separated from the proximally located stent segments.

25. A method as in claim 24, wherein the plurality of adjacent stent segments are introduced into a blood vessel.

26. A method as in claim 24, wherein the plurality includes at least 3 stent segments.

27. Method as in claim 26, wherein at least two stent segments are deployed simultaneously.

28. A method as in claim 24, wherein at least some of the plurality of stent segments are unattached to each other prior to separating.

29. A method as in claim 24, wherein at least some of the plurality of stent segments are attached prior to separating.

30. A method as in claim 24, wherein selecting comprises aligning a distal-most stent segment with a distal end of the region to be treated and identifying a first proximally located stent segment which is aligned with a proximal end of the region, wherein the stent segments distal to the first proximally located stent segment are selected for delivery to the body lumen.

31. A method as in claim 30, wherein selecting is performed under fluoroscopic imaging.

32. A method as in claim 24, wherein axially separating comprises engaging the stent segment which is located immediately proximally of the selected segment(s) with a separator and drawing the separator proximally.

33. A method as in claim 32, wherein the separator comprises a tube and engaging comprises positioning a grip structure near a distal end of the tube over the immediately proximal stent segment.

34. A method as in claim 33, wherein the separator tube comprises a tube that surrounds the proximally located stent segments.

35. A method as in claim 24, wherein deploying comprises inflating a deployment balloon to radially expand the selected stent segment(s).

36. A method as in claim 35, wherein the proximally located stent segments are radially constrained while the selected stent segment(s) are being radially expanded.

37. A method as in claim 34, wherein the stent segments are uncovered by the tube during introduction to the region of the body lumen. A method as in claim 25, further comprising releasing a therapeutic agent from the stent segments after deployment of said segments in the body lumen.

38. A method for selectively delivering stent segments to a treatment region in a blood vessel, said method comprising:
positioning a deployment catheter through the blood vessel to the treatment region, wherein a plurality of adjacent stent segments are positioned over a balloon or catheter;
advancing a separator tube over one or more proximally positioned stent segment(s);
engaging a grip structure on the separator tube against a distal most of the proximally positioned stent segments;
pulling the separator tube proximally to separate the proximally positioned stent segments from the remaining distally positioned stent segment(s); and
inflating the balloon to deploy the distally positioned stent segment(s) while the proximally positioned stent segments remain covered by the separator tube.

39. A method as in claim 38, wherein the plurality includes at least 3 stent segments.

40. A method as in claim 39, wherein at least two distally positioned stent segments are deployed simultaneously.

41. A method as in claim 38, wherein at least some of the plurality of stent segments are unattached to each other prior to separating.

42. A method as in claim 38, wherein at least some of the plurality of stent segments are attached to each other prior to separating.

43. A method as in claim 38, further comprising aligning the distal most stent segment with the distal end of the region to be treated, wherein the separator tube is advanced until the grip structure is engaged against a stent segment which lies immediately proximally of a proximal end of the region to be treated.

44. A method as in claim 43, wherein aligning is performed under fluoroscopic imaging.

45. A method as in claim 38, wherein the stent segments are uncovered by the separator tube during the positioning of the deployment catheter.

46. A method as in claim 38, further comprising releasing a therapeutic agent from the stent segments to inhibit restenosis in the blood vessel.
